(19) United States
(12) Patent Application Publication
(10) Pub. No.: US 2009/0088412 A1
(43) Pub. Date: Apr. 2, 2009

(54) COMPOSITION FOR TREATING CANCER CELLS AND PREPARATION METHOD THEREOF

(75) Inventors: Yang-Chang WU, Kaohsiung City (TW); Fang-Rong CHANG, Kaohsiung City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: KAOHSIUNG MEDICAL UNIVERSITY, Kaohsiung City (TW)

(21) Appl. No.: 12/169,927

(22) Filed: Jul. 9, 2008

(30) Foreign Application Priority Data
Oct. 2, 2007 (TW) 096136960

(51) Int. Cl. A61K 31/58; C07J 17/00; A61P 35/00; C07J 71/00

(52) U.S. Cl. 514/175; 540/115; 540/79; 514/172

(57) ABSTRACT

A composition for treating cancer cells and a preparation method therefore is provided. The composition includes novel withanolide compounds derived from a Solanaceae plant, which the novel withanolide compounds have the cytotoxicity to the cancer cells.

### FIELD OF THE INVENTION

The present invention relates to a composition for treating cancer cells and a preparation method therefor, and more particularly to a composition including an extract extracted from a Tubocapsicum spp. and a preparation method therefor.

### BACKGROUND OF THE INVENTION

Although the modern medicine is well-developed, the treatment effects to some thorny diseases such as cancer, cardiovascular disease, and AIDS are still not satisfactory. Besides, the treatment drugs used in western medicine usually have many side effects, and hence the interests in the substitute therapies including the herbal medicines have increased greatly. Many extracts of the plants and the derivatives derived therefrom, such as vincristine & vinblastine, camptothecin and taxol etc. are widely used in clinical tumor treatment.

Tubocapsicum anomalum (Franch. & Sav.) Makino belongs to Tubocapsicum (Solanaceae), and is mainly distributed in the Southeast Asia around 0-2000 meters over the sea level. It is a folk medicine in Taiwan for treating gonorrhea, dysentery, nephritis and swells. However, the cytotoxic effect of Tubocapsicum anomalum Makino is still indefinite.

In order to overcome the drawbacks in the prior art, a composition including an extract extracted from a Tubocapsicum spp and a preparation method therefor are provided. The particular composition in the present invention not only solves the problems described above, but also is easy to be applied. Thus, the invention has the utility for the industry.

### SUMMARY OF THE INVENTION

In accordance with the present invention, there is provided a cytotoxic composition, which includes at least a withanolide compound having a structure selected from the following formulas:

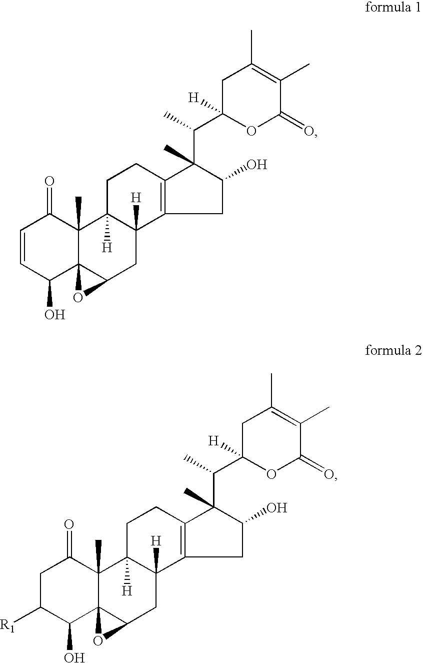

wherein R₁ is OH or C1-C4 alkoxy groups,

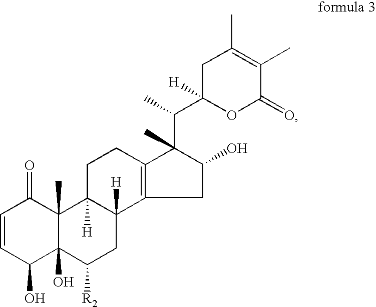

wherein R₂ is H, OH, halogen or C1-C4 alkoxy groups,

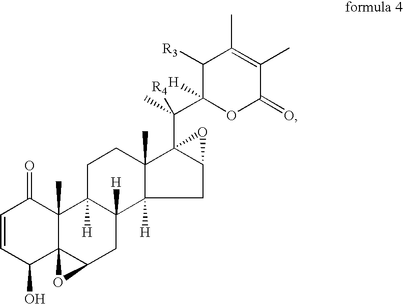

wherein each of R₃ and R₄ is selected from H or OH,

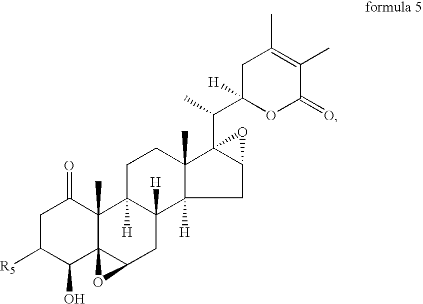

wherein R₅ is OH or C1-C4 alkoxy groups, and

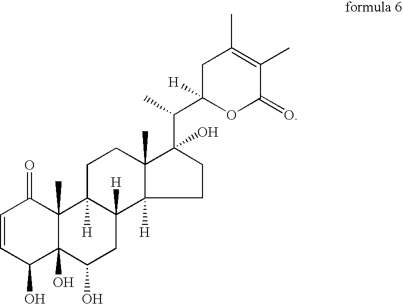

Preferably, the withanolide compound is extracted from a Solanaceae plant.

Preferably, the Solanaceae plant is a Tubocapsicum anomalum Makino.

Preferably, the cytotoxic composition further includes a pharmaceutically acceptable carrier or an excipient.

Preferably, the cytotoxic composition is used for treating a cancer.

Preferably, the cancer is a lung cancer, a liver cancer, or a breast cancer.

In accordance with another aspect of the invention, there is provided a cytotoxic composition, includes a withanolide compound having a structure selected from the following formulas:

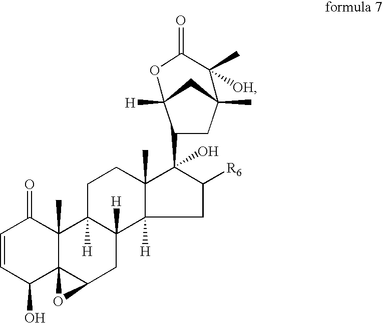

wherein R₆ is H or OH,

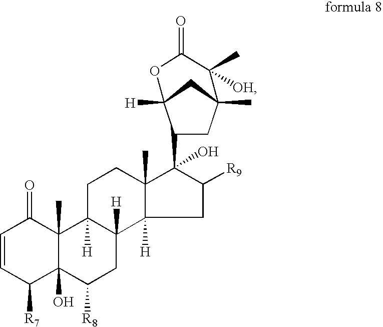

wherein each of R₇ and R₉ is H or OH, and R8 is H, OH or halogen,

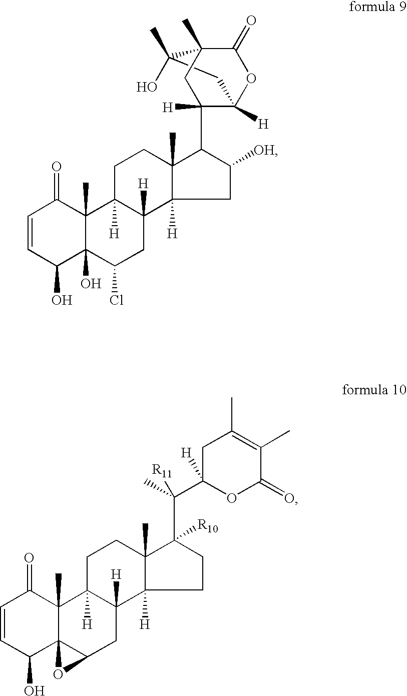

wherein each of R₁₀ and R₁₁ is H or OH,

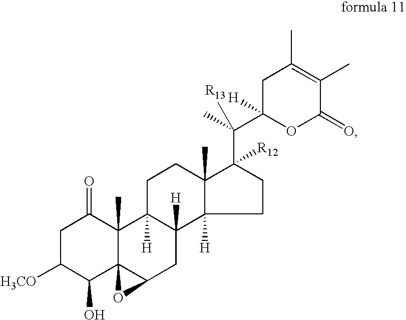

wherein each of R₁₂ and R₁₃ is H or OH, and

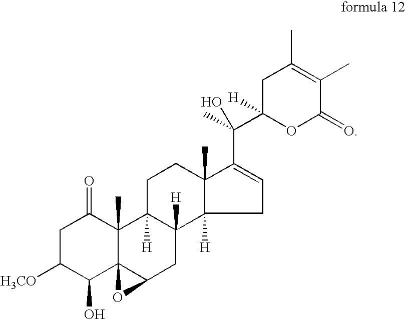

Preferably, the withanolide compound is extracted from a Solanaceae plant.

Preferably, the Solanaceae plant is a Tubocapsicum anomalum.

Preferably, the cytotoxic composition further includes a pharmaceutically acceptable carrier or an excipient.

Preferably, the cytotoxic composition is used for treating a cancer.

Preferably, the cancer is a lung cancer, a liver cancer, or a breast cancer.

In accordance with a further aspect of the present invention, a method for preparation of a withanolide compound is provided. The method includes steps as follows. Firstly, a Tubocapsicum anomalum is provided. Secondly, the Tubocapsicum anomalum is extracted with a first organic solvent to obtain a first extract, and then the first extract is extracted with a second organic solvent to obtain a second extract. Finally, the second extract is isolated to obtain the withanolide compound.

Preferably, the method further includes a step of extracting the second extract with a third organic solvent to obtain a third extract.

Preferably, the third organic solvent is an n-butanol.

Preferably, the method further includes a step of isolating the third extract to obtain the withanolide compound.

Preferably, the withanolide compound is isolated from the third extract by a chromatography method.

Preferably, the first organic solvent is a methanol.

Preferably, the second organic solvent is an ethyl acetate.

Preferably, the withanolide compound is isolated from the second extract by a chromatography method.

The above objects and advantages of the present invention will become more readily apparent to those ordinarily skilled in the art after reviewing the following detailed descriptions and accompanying table, in which:

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

The composition provided in the present invention includes a withanolide compound that is extracted from Tubocapsicum anomalum. The preferred embodiments of the preparation methods for the withanolide compound will be introduced as follows.

### EXAMPLE 1

The stems and leaves of the collected Tubocapsicum anomalum weighted 2.5 Kg are extracted with 3-liter methanol repeatedly five times, to obtain the first extracts. The first extracts are concentrated under reduced pressure to obtain a dark green, viscous residue, which is further partitioned between EtOAc/H₂O to yield EtOAc and H₂O extracts respectively. The EtOAc extracts are concentrated under reduced pressure to obtain the second extracts, and the H₂O extracts are further partitioned between n-BuOH/H₂O to yield n-BuOH and H₂O extracts respectively. The n-BuOH extracts are concentrated under reduced pressure to further obtain the third extracts. The residue from the EtOAc extracts are further separated by a column chromatography (Si gel, 230-400 mesh, 5×20 cm), and eluted with a gradient of n-Hexane→n-Hexane/CHCl₃→CHCl₃→CHCl₃/MeOH→MeOH on a thin-layer chromatography to give 16 fractions.

The fraction 8 (Fr. 8, 529.8 mg) is chromatographed from the EtOAc extracts using CHCl₃/MeOH (20:1) as eluents, and recrystallized from MeOH to give Tubocapsanolide A (42.5 mg, CHCl₃:MeOH=20:1, R_{f}=0.5).

The fraction 11 (Fr. 11, 165.2 mg) is chromatographed from the EtOAc extracts using CHCl₃/MeOH (15:1) as eluents, and recrystallized from MeOH to give Tubocapsenolide A (101.1 mg, CHCl₃:MeOH=25:1, R_{f}=0.2).

The fraction 12 (Fr. 12, 120.1 mg) is chromatographed from the EtOAc extracts using CHCl₃/MeOH (15:1) as eluents, and recrystallized from MeOH to give Anomanolide C (65.3 mg, CHCl₃:MeOH=10:1, R_{f}=0.5).

The mother liquors of fractions 8 to 12 are combined to obtain the sample 1 (TAEw, 400 mg), and then partitioned with H₂O/MeOH/CHCl₃ (1:4:5) to yield MeOH fraction (TAEWM, 250 mg) and CHCl₃ fraction (TAEWC, 70 mg). The fraction TAEWM is further separated by HPLC (ODS 250×21.2 mm, MeOH:H₂O=65:35, PDA-detector: UV-230 nm, flow rate: 3 ml/min) to afford 6 fractions (1-1 to 1-6). The fraction 1-4 is Tubocapsenolide A (41 mg, ODS 250×21.2 mm, MeOH:H₂O=65:35, flow rate: 3 ml/min, R.t.=28 min), and the fraction 1-6 is Tubocapsanolide A, (10 mg, ODS 250×21.2 mm, MeOH:H₂O=65:35, flow rate: 3 ml/min, R.t.=35 min).

The fraction 1-2 is further separated by HPLC (ODS 250×10 mm, MeOH:H₂O=50:50, PDA-detector: UV-230 nm, flow rate: 2 ml/min) to afford 6 fractions (1-2-1 to 1-2-6).

The fraction 1-2-3 (Fr. 1-2-3, 8.63 mg) is separated by HPLC (ODS 250×10 mm, MeOH:H₂O=45:55, PDA-detector: UV-230 nm, flow rate: 2 ml/min) to afford the numerous fractions, wherein the fraction 1-2-3-2 is Anomanolide E (3.5 mg, ODS 250×4.6 mm, MeOH:H₂O=45:55, flow rate: 1 ml/min, R.t.=11.4 min).

The fraction 1-2-4 (23 mg) is separated by HPLC (ODS 250×10 mm, MeOH:H₂O=45:55, PDA-detector: UV-230 nm, flow rate: 2 ml/min) to afford the numerous fractions, wherein the fraction 1-2-4-3 is Anomanolide A (14 mg, ODS 250×4.6 mm, MeOH:H₂O=45:55, flow rate: 1 ml/min, R.t.=12.4 min).

The fraction 1-3 (32 mg) is separated by HPLC (ODS 250×10 mm, MeOH:H₂O=45:55, PDA-detector: UV-230 nm, flow rate: 2 ml/min) to afford the numerous fractions, wherein the fraction 1-3-4 is Tubocapsenolide B (2.3 mg, ODS 250×10 mm, MeOH:H₂O=45:55, flow rate: 2 ml/min, R.t.=62.5 min).

The fraction 1-5 (26 mg) is dissolved in MeOH and stay a few days, and a white solid would be recrystallized. The liquor is filtered through the cotton to obtain Tubocapsenolide D (20 mg, ODS 250×4.6 mm, MeOH:H₂O=50:50, flow rate: 1 ml/min, R.t.=24 min).

The n-BuOH extracts (5.9) are further partitioned between CHCl₃/H₂O (1:1) to yield CHCl₃ extracts (TABC, 1.38 g) and H₂O extracts (TABH 4.2 g) respectively. The CHCl₃ extracts (TABC) are further separated by a column chromatography (Sephadex, LH-20, 4.5×50 cm), and eluted with MeOH on a thin-layer chromatography to give 7 fractions.

The fraction 4 (276.5 mg) is further separated by a column chromatography (Si gel, 230-400 mesh, 2.5×27 cm), and eluted with CHCl₃ increasing the polarity gradually to MeOH to give 21 fractions.

The fraction 4-5 (17.15 mg) is separated by HPLC (ODS 250×10 mm, MeOH:H₂O=75:25, UV-detector: UV-230 nm, flow rate: 2 ml/min) to afford numerous fractions, wherein the fraction 4-05 is Tubocapsanolide C (2.7 mg, ODS 250×10 mm, MeOH:H₂O=75:25, flow rate: 2 ml/min, R.t.=42.5 min).

The fraction 4-10 (6.22 mg) is separated by HPLC (ODS 250×4.6 mm, MeOH:H₂O=70:30, PDA-detector: UV-230 nm, flow rate: 1 ml/min) to afford numerous fractions, wherein the fraction 4-10 is Tubocapsenolide E (2.7 mg, ODS 250×4.6 mm, MeOH:H₂O=70:30, flow rate: 1 ml/min, R.t.=14.3 min).

The fraction 4-17 (11.1 mg) is separated by HPLC (ODS 250×4.6 mm, MeOH:H₂O=70:30, PDA-detector: UV-230 nm, flow rate: 1 ml/min) to afford numerous fractions, wherein the fraction 4-17-1 is Tubocapsenolide F (5.12 mg, ODS 250×4.6 mm, MeOH:H₂O=70:30, flow rate: 1 ml/min, R.t.=17.5 min).

The fraction 5 (278.3 mg) is separated by a column chromatography (Si gel, 230-400 mesh, 2×25 cm), and eluted with CHCl₃/MeOH (150:1) increasing the polarity gradually to MeOH to give 22 fractions, wherein the fraction 5-02 is Tubocapsanolide E (15.16 mg, CHCl₃:MeOH=20:1, R_{f}=0.5).

The fraction 5-03 (2.47 mg) is separated by HPLC (ODS 250×10 mm, MeOH:H₂O=70:30, PDA-detector: UV-230 nm, flow rate: 1 ml/min) to afford the numerous fractions, wherein the fraction 5-03 is Tubocapsanolide D (1.2 mg, ODS 250×10 mm, MeOH:H₂O=70:30, flow rate: 2 ml/min, R.t.=17.4 min).

The fraction 5-07 (39.38 mg) is separated by HPLC (ODS 250×4.6 mm, MeOH:H₂O=70:30, PDA-detector: UV-230 nm, flow rate: 1 ml/min) to afford the numerous fractions, wherein the fraction 5-07 is Anomanolide B (8.3 mg, ODS 250×4.6 mm, MeOH:H₂O=70:30, flow rate: 1 ml/min, R.t.=6.1 min).

The fraction 5-09 (15.7 mg) is separated by HPLC (ODS 250×10 mm, MeOH:H₂O=60:40, PDA-detector: UV-230 nm, flow rate: 2 ml/min) to afford the numerous fractions, wherein the fraction 5-09-2 is Tubocapsanolide B (8.3 mg, ODS 250×10 mm, MeOH:H₂O=60:40, flow rate: 2 ml/min, R.t.=40.8 min).

The fraction 5-11 (19.66 mg) is separated by HPLC (ODS 250×10 mm, MeOH:H₂O=65:35, PDA-detector: UV-230 nm, flow rate: 2 ml/min) to afford the numerous fractions, wherein the fraction 5-11 is Tubocapsenolide G (11.53 mg, ODS 250×4.6 mm, MeOH:H₂O=65:35, flow rate: 1 ml/min, R.t.=4.1 min).

The fraction 5-18 (15.15 mg) is separated by HPLC (ODS 250×10 mm, MeOH:H₂O=65:35, PDA-detector: UV-230 nm, flow rate: 2 ml/min) to afford the numerous fractions, wherein the fraction 5-18-1 is Tubocapsenolide C (6.72 mg, ODS 250×10 mm, MeOH:H₂O=65:35, flow rate: 2 ml/min, R.t.=17.6 min).

The fraction 6 (108.73 mg) is separated by a column chromatography (Si gel, 230-400 mesh, 2×22 cm), and eluted with CHCl₃/MeOH (50:1) increasing the polarity gradually to MeOH to give 16 fractions, wherein the fraction 6-12 is Anomanolide D (33.5 mg, CHCl₃:MeOH=10:1, R_{f}=0.4).

The fraction 6-14 (2.05 mg) is separated by HPLC (ODS 250×4.6 mm, MeOH:H₂O=50:50, PDA-detector: UV-230 nm, flow rate: 1 ml/min) to afford the numerous fractions, wherein the fraction 6-14-4 is Tubonolide A (1.15 mg, ODS 250×4.6 mm, MeOH:H₂O=50:50, flow rate: 1 ml/min, R.t.=13.8 min).

### EXAMPLE 2

The roots of the fresh Tubocapsicum anomalum are extracted with methanol (MeOH) successively five times (24 hours each), to obtain extracting liquor. The extracting liquor is concentrated under reduced pressure and then is partitioned between EtOAc/H₂O to yield EtOAc and H₂O extracts respectively. The H20 extracts are further partitioned between n-BuOH/H₂O to yield n-BuOH and H₂O extracts respectively, and the EtOAc extracts are partitioned between MeOH/n-hexane to yield MeOH and n-hexane extracts respectively. The MeOH extracts are separated by column chromatography (Sephadex LH-20, 1.6×28 cm), and eluted with MeOH to give 5 fractions. The fraction 3 (807 mg) is further separated by column chromatography (Si gel, 230-400 mesh, 5×20 cm), and eluted with a gradient of CHCl₃→CHCl₃/MeOH→MeOH on a thin-layer chromatography to give 28 fractions.

The fraction 3-3 is separated by HPLC (ODS 250×10 mm, MeOH:H₂O=65:35, UV/VIS-detector: UV-230 nm, flow rate: 2 ml/min) to afford 8 fractions. The fraction 3-3-7 is iso-tubocapsanolide G (ODS 250×10 mm, MeOH:H₂O=65:35, UV/VIS-detector: UV-230 nm, flow rate: 2 ml/min, R.t.=34 min).

The fraction 3-3-3 is separated by HPLC (ODS 250×10 mm, MeOH:H₂O=65:35, UV/VIS-detector: UV-230 nm, flow rate: 2 ml/min) and recycle to afford the fraction 3-3-3-1, which is 20-hydroxy-tubocapsanolide A (ODS 250×10 mm, MeOH:H₂O=65:35, UV/VIS-detector: UV-230 nm, flow rate: 2 ml/min, R.t.=17 min), and the fraction 3-3-3-2 is 20-hydroxy-tubocapsanolide G (ODS 250×10 mm, MeOH:H₂O=65:35, UV/VIS-detector: UV-230 nm, flow rate: 2 ml/min, R.t.=18 min).

The fraction 3-3-4 is separated by HPLC (ODS 250×10 mm, MeOH:H₂O=65:35, UV/VIS-detector: UV-230 nm, flow rate: 2 ml/min) and recycle to afford the fraction 3-3-4-1-2, which is Tubocapsanolide H (ODS 250×10 mm, MeOH:H₂O=65:35, UV/VIS-detector: UV-230 nm, flow rate: 2 ml/min, R.t.=24 min), the fraction 3-3-4-2 is Tubocapsanolide F (ODS 250×10 mm, MeOH:H₂O=65:35, UV/VIS-detector: UV-230 nm, flow rate: 2 ml/min, R.t.=25 min), and the fraction 3-3-4-4 is Tubocapsanolide G (ODS 250×10 mm, MeOH:H₂O=65:35, UV/VIS-detector: UV-230 nm, flow rate: 2 ml/min, R.t.=26 min).

The fraction 3-8 is separated by HPLC (ODS 250×10 mm, MeOH:H₂O=65:35, UV/VIS-detector: UV-230 nm, flow rate: 2 ml/min) to afford the numerous fraction, wherein the fraction 3-8-2 is 23-Hydroxy-tubocapsanolide A (ODS 250×10 mm, MeOH:H₂O=65:35, UV/VIS-detector: UV-230 nm, flow rate: 2 ml/min, R.t.=13.5 min).

The fraction 3-10 (126.8 mg) is separated by HPLC (Si gel, 230˜400 mesh, 3×15 cm), and eluted with a CHCl₃/MeOH (20:1) to give 3 fractions. The fraction 3-10-3 (11 mg) is separated by HPLC (ODS 250×10 mm, MeOH:H₂O=60:40, UV/VIS-detector: UV-230 nm, flow rate: 2 ml/min) to afford the numerous fractions, wherein the fraction 3-10-3-2 is Anomanolide F (ODS 250×10 mm, MeOH:H₂O=60:40, UV/VIS-detector: UV-230 nm, flow rate: 2 ml/min, R.t.=14.5 min).

The preparation methods for the withanolide compounds according to the preferred embodiments of the present invention are described above. Based on such preparation methods, 25 all new withanolide compounds are extracted from Tubocapsicum anomalum. After further analyzing respectively, the character of each compound are described as follows.

The physical and chemical characters of compound Tubocapsenolide A (1): m.p.: 223-225° C.; UV (MeOH): λₘₐₓ 212, 228 nm; IR: νₘₐₓ 3380, 2921, 1677, 1380, 1130 cm⁻¹; ¹NMR (400 MHz, pyridine-d₅): δ 6.47 (1H, d, J=10.0 Hz, H-2), 7.27 (1H, dd, J=10.0, 6.4 Hz, H-3), 4.05 (1H, d, J=6.4 Hz, H-4), 3.37 (1H, s(br), H-6), 1.39 (1H, m, H-7a), 2.33 (1H, m, H-7b), 2.35 (1H, m, H-8), 1.37 (1H, m, H-9), 1.26 (1H, ddd, J=12.4, 4.8, 4.0 Hz, H-11a), 2.04 (1H, m, H-11b), 1.77 (1H, m, H-12a), 2.04 (1H, m, H-12b), 2.48 (2H, m, H-15a,b), 4.45 (1H, t, J=8.0 Hz, H-16), 1.21 (3H, s, CH₃-18), 1.81 (3H, s, CH₃-19), 2.58 (1H, qd, J=7.2, 3.6 Hz, H-20), 1.17 (3H, d, J=7.2 Hz, CH₃-21), 5.17 (1H, ddd, J=12.8, 3.6, 3.2 Hz, H-22), 2.47 (1H, m, H-23a), 2.30 (1H, m, H-23b), 1.87 (3H, s, CH₃-27), 1.77 (3H, s, CH₃-28). EI-MS m/z (rel. int.): 469 [M+H]⁺. HR-ESI-MS: m/z 469.2594 [M+H]⁺ (C₂₈H₃₇O₆, 469.2585).

The physical and chemical characters of compound Tubocapsenolide B (2): m.p.: 133˜135° C.; UV (MeOH): λₘₐₓ 208, 228 nm; IR: νₘₐₓ 3421, 2921, 1691, 1380, 1130 cm⁻; ¹H NMR (400 MHz, pyridine-d₅): δ 2.50 (1H, m, H-2a), 2.79 (1H, J=15.2, 8.0, 7.6 Hz, H-2b), 1.96 (1H, m, H-3a), 2.22 (1H, m, H-3b), 3.54 (1H, dd, J=4.0, 3.6 Hz, H-4), 3.24 (1H, s(br), H-6), 1.25 (1H, m, H-7a), 2.35 (1H, ddd, J=14.4, 4.0, 2.0 Hz, H-7b), 2.10 (1H, m, H-8), 1.40 (1H, dd, J=12.4, 11.2 Hz, H-9), 1.06 (1H, m, H-11a), 2.04 (1H, m, H-11b), 1.77 (2H, m, H-12a,b), 2.43 (2H, m, H-15a,b), 4.12 (1H, t, J=7.6 Hz, H-16), 1.12 (3H, s, CH₃-18), 1.30 (3H, s, CH₃-19), 2.16 (1H, qd, J=7.2, 3.2 Hz, H-20), 0.98 (3H, d, J=7.2 Hz, CH₃-21), 4.35 (1H, ddd, J=12.4, 7.2, 3.2 Hz, H-22), 2.48 (1H, m, H-23a), 2.24 (1H, m, H-23b), 1.87 (3H, s, CH₃-27), 1.94 (3H, s, CH₃-28). EI-MS m/z (rel. int.): 471 [M+H]⁺. HR-ESI-MS: m/z 493.2568 [M+Na]⁺ (calculated for C₂₈H₃₈O₆Na, 493.2561).

The physical and chemical characters of compound Tubocapsenolide C (3): m.p.: 226˜228° C.; UV (MeOH): λₘₐₓ 208, 228 nm; IR: νₘₐₓ 3411, 2919, 1681, 1135, 1060 cm⁻¹; ¹H NMR (400 MHz, CD₃OD): δ 2.58 (1H, dd, J=12.4, 2.8 Hz, H-2a), 3.15 (1H, dd, J=12.4, 6.0 Hz, H-2b), 4.06 (1H, dt, J=6.0, 2.8 Hz, H-3), 3.31 (1H, d, J=2.8 Hz, H-4), 3.28 (1H, s(br), H-6), 1.41 (1H, d, J=10.8, 2.4, 1.2 Hz, H-7a), 2.32 (1H, ddd, J=10.8, 2.4, 1.2 Hz, H-7b), 2.07 (1H, br, H-8), 1.44 (1H, ddd, J=9.2, 8.0, 1.2 Hz, H-9), 1.05 (1H, dd, J=10.0, 4.0 Hz, H-11a), 1.68 (1H, dd, J=10.0, 1.2 Hz, H-11b), 1.85 (1H, m, H-12a), 2.07 (1H, m, H-12b), 2.20 (1H, m, H-15a), 2.38 (1H, dd, J=12.8, 7.2 Hz, H-15b), 4.10 (1H, t, J=6.8 Hz, H-16), 1.09 (3H, s, CH₃-18), 1.20 (3H, s, CH₃-19), 2.20 (1H, m, H-20), 1.02 (3H, d, J=6.0 Hz, CH₃-21), 4.70 (1H, ddd, J=10.4, 3.2, 2.4 Hz, H-22), 2.54 (1H, dd, J=12.4, 10.4 Hz, H-23a), 2.24 (1H, dd, J=12.4, 2.4 Hz, H-23b), 1.96 (3H, s, CH₃-27), 1.82 (3H, s, CH₃-28). EI-MS m/z (rel. int.): 487 [M+H]⁺. HR-ESI-MS: m/z 509.2516 [M+Na]⁺ (calculated for C₂₈H₃₈O₇Na, 509.2510).

The physical and chemical characters of compound Tubocapsenolide D (4): m.p.: 124˜126° C.; UV (MeOH): λₘₐₓ 210, 228 nm; IR: νₘₐₓ 3409, 2921, 1691, 1384, 1135, 1097 cm⁻¹; ¹H NMR (400 MHz, CD₃OD): δ 2.63 (1H, dd, J=14.8, 4.0 Hz, H-2a), 3.14 (1H, dd, J=14.8, 5.2 Hz, H-2b), 3.75 (1H, ddd, J=4.4, 4.0, 3.6 Hz, H-3), 3.51 (1H, d, J=3.2 Hz, H-4), 3.30 (1H, s(br), H-6), 1.25 (1H, m, H-7a), 2.32 (1H, ddd, J=14.4, 4.0, 2.4 Hz, H-7b), 2.07 (1H, m, H-8), 1.44 (1H, ddd, J=10.8, 10.0, 2.0 Hz, H-9), 1.05 (1H, dd, J=12.0, 4.0 Hz, H-11a), 2.02 (1H, m, H-11b), 1.83 (2H, m, H-12a,b), 2.42 (2H, m, H-15a,b), 4.13 (1H, dd, J=8.4, 7.6 Hz, H-16), 1.12 (3H, s, CH₃-18), 1.30 (3H, s, CH₃-19), 2.17 (1H, qd, J=7.2, 6.8 Hz, H-20), 0.98 (3H, d, J=7.2 Hz, CH₃-21), 4.37 (1H, ddd, J=12.8, 6.4, 3.2 Hz, H-22), 2.48 (1H, m, H-23a), 2.30 (1H, dd, J=17.6, 3.2 Hz, H-23b), 1.86 (3H, s, CH₃-27), 1.94 (3H, s, CH₃-28), 3.34 (3H, s, OCH₃-1′). FAB-MS m/z (rel. int.): 501 [M+H]⁺, 523 [M+Na]⁺. HR-ESI-MS: m/z 523.2672 [M+Na.]⁺ (calculated C₂₉H₄₀O₇Na, 523.2666).

The physical and chemical characters of compound Tubocapsenolide E (5): m.p.: 104˜106° C.; UV (MeOH): λₘₐₓ 210, 228 nm; IR: νₘₐₓ 3413, 2917, 1702, 1687, 1394, 1132, 1091 cm⁻; ¹H NMR (400 MHz, CDCl₃): δ 2.62 (1H, dd, J=14.4, 4.0 Hz, H-2a), 3.14 (1H, dd, J=14.4, 5.6 Hz, H-2b), 3.82 (1H, ddd, J=4.4, 4.4, 4.0 Hz, H-3), 3.49 (1H, m, H-4), 3.29 (1H, s(br), H-6), 1.23 (1H, J=14.4, 1.2 Hz, H-7a), 2.33 (1H, ddd, J=14.4, 3.2, 2.4 Hz, H-7b), 2.07 (1H, m(br), H-8), 1.42(1H, ddd, J=10.8, 10.0, 1.6 Hz, H-9), 1.05(1H, dd, J=12.0, 4.4 Hz, H-11a), 2.02 (1H, m(br), H-11b), 1.82 (2H, m, H-12a,b), 2.42 (2H, m, H-15a,b), 4.13 (1H, t, J=8.0 Hz, H-16), 1.12 (3H, s, CH₃-18), 1.29 (3H, s, CH₃-19), 2.17 (1H, qd, J=7.2, 6.8 Hz, H-20), 0.98 (3H, d, J=7.2 Hz, CH₃-21), 4.37 (1H, ddd, J=12.8, 6.4, 3.2 Hz, H-22), 2.44 (1H, m, H-23a), 2.20 (1H, dd, J=17.2, 2.4 Hz, H-23b), 1.85 (3H, s, CH₃-27), 1.93 (3H, s, CH₃-28), 3.34 (3H, s, OCH₃-1′), 3.39 (1H, td, J=9.2, 6.4 Hz, H-1′a), 3.50 (1H, td, J=9.2, 6.4 Hz, H1′b), 1.48 (2H, m, H-2′a,b), 1.30 (2H, m, H-3′a,b), 0.88 (3H, t, J=8.8 Hz, CH₃-4′). ESI-MS m/z (rel. int.): 543 [M+H]⁺. HR-ESI-MS: m/z 565.3193 [M+Na]⁺ (calculated for C₃₂H₄₆O₇Na, 565.3136).

The physical and chemical characters of compound Tubocapsenolide F (6): m.p.: 214˜216° C.; UV (MeOH): λₘₐₓ 212, 228 nm; IR: νₘₐₓ 3424, 2927, 1679, 1380, 113.2 cm⁻¹; ¹H NMR (400 MHz, C₅D₅N): δ 6.28 (1H, dd, J=10.0, 2.0 Hz, H-2), 6.91 (1H, dd, J=10.0, 2.0 Hz, H-3), 5.37 (1H, s(br), H-4), 3.81 (1H, dd, J=12.8, 4.0 Hz, H-6), 2.01 (1H, m, H-7a), 2.99 (1H, dt, J=13.2, 4.0 Hz, H-7b), 2.30 (1H, m, H-8), 1.94 (1H, m, H-9), 1.26 (1H, m, H-11a), 2.01 (1H, m, H-11b), 1.66 (1H, m, H-12a), 1.85 (1H, m, H-12b), 2.38 (2H, m, H-15a,b), 4.37 (1H, dd, J=8.0, 7.6 Hz, H-16), 1.18 (3H, s, CH₃-18), 1.73 (3H, s, CH₃-19), 2.55 (1H, qd, J=7.6, 3.2 Hz, H-20), 1.12 (3H, d, J=7.6 Hz, CH₃-21), 5.17 (1H, m, H-22), 2.45 (1H, m, H-23a), 2.30 (1H, dd, J=14.4, 2.4 Hz, H-23b), 1.78 (3H, s, CH₃-27), 1.66 (3H, s, CH₃-28). HR-ESI-MS: m/z 509.2520 [M+Na]⁺ (calculated for C₂₈H₃₈O₇Na, 509.2510).

The physical and chemical characters of compound Tubocapsenolide G (7): m.p.: 264˜266° C.; UV (MeOH): λₘₐₓ 216 nm; IR: νₘₐₓ 3478, 2947, 1723, 1676, 1379, 1126 cm⁻¹; ¹H NMR (400 MHz, CDCl₃): δ 6.01 (1H, dd, J=10.4, 2.0 Hz, H-2), 6.47 (1H, dd, J=10.4, 2.0 Hz, H-3), 5.04 (1H, t, J=2.0 Hz, H-4), 4.45 (1H, dd, J=10.4, 3.6 Hz, H-6), 1.85 (1H, J=10.4, 10.4 Hz, H-7a), 2.51 (1H, ddd, J=10.4, 3.6, 3.6 Hz, H-7b), 2.33 (1H, m, H-8), 1.50 (1H, td, J=9.6, 1.6 Hz, H-9), 1.28 (1H, m, H-lla), 2.01 (1H, m, H-11b), 1.63 (1H, m, H-12a), 1.85 (1H, m, H-12b), 2.40 (1H, m, H-15a), 2.55 (1H, m, H-15b), 4.09 (1H, dd, J=7.6, 6.0 Hz, H-16), 1.06 (3H, s, CH₃-18), 1.20 (3H, s, CH₃-19), 2.15 (1H, qd, J=7.2, 7.2 Hz, H-20), 0.96 (3H, d, J=7.2 Hz, CH₃-21), 4.37 (1H, ddd, J=12.8, 7.2, 2.4 Hz, H-22), 2.42 (1H, m, H-23a), 2.22 (1H, dd, J=18.0, 2.4 Hz, H-23b), 1.86 (3H, s, CH₃-27), 1.92 (3H, s, CH₃-28). ESI-MS m/z (rel. int.): 505 [M+H]⁺. HR-ESI-MS: m/z 527.2177 [M+Na]⁺ (calculated for C₂₈H₃₇ClO₆Na, 527.2171).

The physical and chemical characters of compound Tubocapsanolide A (8): m.p.: 233˜235° C.; UV (MeOH): λₘₐₓ 218 nm; IR: νₘₐₓ 3403, 2918, 1688, 1679, 1380, 1132 cm⁻¹; ¹H NMR (400 MHz, C₅D₅N): δ 6.43 (1H, d, J=9.6 Hz, H-2), 7.23 (1H, dd, J=9.6, 6.0 Hz, H-3), 4.01 (1H, dd, J=6.0, 4.6 Hz, H-4), 3.20 (1H, s(br), H-6), 1.17 (1H, ddd, J=14.8, 11.2, 1.2 Hz, H-7a), 2.01 (1H, m, H-7b), 1.63 (1H, ddd, J=11.2, 11.2, 4.0 Hz, H-8), 0.98 (1H, ddd, J=11.6, 11.2, 4.0 Hz, H-9), 1.57(1H, m, H-11a), 2.01(1H, m, H-11b), 1.40(1H, m, H-12a), 1.60 (1H, m, H-12b), 1.24 (1H, ddd, J=12.0, 11.6, 6.4 Hz, H-14), 1.12 (1H, dd, J=12.4, 12.0 Hz, H-15a), 1.86 (1H, dd, J=12.4, 6.4 Hz, H-15b), 3.59 (1H, s(br), H-16), 0.89 (3H, s, CH₃-18), 1.81 (3H, s, CH₃-19), 2.45 (1H, qd, J=8.8, 6.8 Hz, H-20), 1.02 (3H, d, J=6.8 Hz, CH₃-21), 3.91 (1H, ddd, J=12.8, 8.8, 3.2 Hz, H-22), 2.22 (1H, dd, J=17.6, 12.4 Hz, H-23a), 2.08 (1H, dd, J=17.6, 3.2 Hz, H-23b), 1.92 (3H, s, CH₃-27), 1.73 (3H, s, CH₃-28). FAB-MS m/z (rel. int.): 469 [M+Na]⁺. HR-ESI-MS: m/z 469.2594 [M+Na]⁺(calculated for C₂₈H₃₆O₆Na, 469.2585).

The physical and chemical characters of compound 20-Hydroxytubocapsanolide A (9): m.p.: 245˜247° C.; UV (MeOH): λₘₐₓ 214 nm; IR.: νₘₐₓ 3439, 2922, 2856, 1705, 1380, 1236, 1026, 750 cm⁻¹; ¹H NMR (400 MHz, C₅D₅N): δ 6.46 (1H, d, J=9.5 Hz, H-2), 7.22 (1H, dd, J=6.0 Hz H-3), 4.00 (1H, d, J=6.0 Hz, H-4), 3.21 (1H, br s, H-6), 1.16 (1H, m, H-7a), 2.00 (1H, qd, J=15.0, 2.0 Hz , H-7b), 1.66 (1H, m, H-8), 0.87 (1H, m, H-9), 1.60 (1H, m, H-11a), 1.96 (1H, m, H-11b), 1.85 (1H, m, H-12a), 1.93 (1H, m, H-12b), 1.37 (1H, td, J=6 Hz, H-14), 1.10 (1H, td, J=13.0, 12.0 Hz, H-15a), 1.77 (1H, m, H-15b), 3.57 (1H, s, H-16), 1.06 (3H, s, H-18), 1.81 (3H, s, H-19), 1.45 (3H, s, H-21), 4.48 (1H, dd, J=12.5, 3.5 Hz, H-22), 2.17 (1H, dd, J=18.0, 3.5 Hz, H-23a), 2.83 (1H, t, J=18.0 Hz, H-23b), 1.91 (3H, s, H-27), 1.74 (3H, s, H-28). ESI-MS m/z (rel. int.): 507 [M+Na]⁺, 507(100). HR-ESI-MS: 507.2361 [M+Na]⁺ (calculated for C₂₈H₃₆O₇Na, 507.2359).

The physical and chemical characters of compound 23-Hydroxytubocapsanolide A (10): m.p.: 223˜225° C.; UV (MeOH): λₘₐₓ 216 nm; IR: νₘₐₓ 3409, 2922, 2848, 1690, 1447, 1380, 753 cm⁻; ¹H NMR (400 MHz, C₅D₅N): δ 6.42 (11H, d, J=9.6 Hz, H-2), 7.21 (11H, dd, J=9.6, 6.4 Hz, H-3), 3.99 (1H, d, J=6.4 Hz, H-4), 3.18 (1H, br s, H-6), 1.16 (1H, m, H-7a), 1.98 (1H, m, H-7b), 1.63 (1H, m, H-8), 0.92 (1H, m, H-9), 2.01 (2H, m, H-11ab), 1.38 (1H, m, H-12a), 1.56 (1H, m, H-12b), 1.22 (1H, m, H-14), 1.13 (1H, m, H-15a), 1.72 (1H, dd, J=12.4, 5.6 Hz H-15b), 3.813 (1H, s, H-16), 0.82 (3H, s, H-18), 1.79 (3H, s, H-19), 2.59 (1H, qd, J=7.2, 6.8 Hz, H-20), 1.15 (3H, d, J=6.8 Hz, H-21), 4.36 (1H, m, H-22), 4.39 (1H, d, H-23), 1.98 (3H, s, H-27), 2.09 (3H, d, J=0.8 Hz, H-28). ESI-MS m/z (rel. int.); 507[M+Na]⁺, 507(100), 413(75), 381(48), 353(12). HR-ESI-MS: 507.2361 [M+Na]⁺ (calculated for C₂₈H₃₆O₇Na, 507.2359).

The physical and chemical characters of compound Tubocapsanolide B (11): m.p.: 225˜227° C.; UV (MeOH): λₘₐₓ 207, 223 nm; IR: νₘₐₓ 3411, 2908, 1698, 1382, 1126 cm⁻¹; ¹H NMR (400 MHz, C₅D₅N: δ 2.97 (11H, dd, J=15.2, 3.2 Hz, H-2a), 3.15 (11H, dd, J=15.2, 8.0 Hz, H-2b), 3.93 (1H, m, H-3), 3.89 (1H, s(br), H-4), 3.36 (1H, s(br), H-6), 1.32 (1H, m, H-7a), 2.11 (1H, m, H-7b), 1.61 (1H, m, H-8), 1.32 (1H, m, H-9), 1.53 (1H, m, H-11a,b), 1.39 (1H, m, H-12a), 1.59 (1H, m, H-12b), 1.28 (1H, m, H-14), 1.14 (1H, dd, J=12.8, 12.0 Hz, H-15a), 1.80(1H, dd, J=12.8, 6.0 Hz, H-15b), 3.60(1H, s(br), H-16), 0.87 (3H, s, CH₃-18), 1.69 (3H, s, CH₃-19), 2.45 (1H, qd, J=8.0, 6.8 Hz, H-20), 1.02 (3H, d, J=6.8 Hz, CH₃-21), 3.93 (1H, m, H-22), 2.22 (1H, dd, J=17.2, 12.0 Hz, H-23a), 2.09 (1H, dd, J=17.2, 3.2 Hz, H-23b), 1.86 (3H, s, CH₃-27), 1.92 (3H, s, CH₃-28), 3.33 (3H, s, OCH₃-1′). HR-ESI-MS: m/z 523.2672 [M+Na]⁺ (calculated for C₂₉H₄₀O₇Na, 523.2676).

The physical and chemical characters of compound Tubocapsanolide C (12): m.p.: 223˜225° C.; UV (MeOH): λₘₐₓ 208, 226 nm; IR: νₘₐₓ 3388, 2931, 1702, 1687, 1380, 1095 cm⁻; ¹H NMR (400 MHz, CDCl₃): δ 2.60 (1H, dd, J=15.2, 3.2 Hz, H-2a), 2.94 (1H, dd, J=15.2, 6.4 Hz, H-2b), 3.77 (1H, ddd, J=6.4, 3.2, 2.8 Hz, H-3), 3.49 (1H, d, J=2.8 Hz, H-4), 3.21 (1H, s(br), H-6), 1.35 (1H, m, H-7a), 2.18 (1H, m, H-7b), 1.44 (1H, m, H-8), 1.19 (1H, td, J=12.4, 8.0 Hz, H-9), 1.46 (1H, m, H-11a,b), 1.43 (1H, m, H-12a), 1.60 (1H, m, H-12b), 1.16 (1H, m, H-14), 1.26 (1H, d, J=12.0 Hz, H-15a), 1.86 (1H, m, H-15b), 3.46 (1H, s(br), H-16), 0.84 (3H, s, CH₃-18), 1.29 (3H, s, CH₃-19), 2.42 (1H, qd, J=8.8, 7.2 Hz, H-20), 0.99 d, J=7.2 Hz, CH₃-21), 3.86 (1H, ddd, J=12.0, 8.8, 3.6 Hz, H-22), 2.30 (1H, dd, J=19.2, 12.0 Hz, H-23a), 2.15 (1H, m, H-23b), 1.86 (3H, s, CH₃-27), 1.92 (3H, s, CH₃-28), 3.39 (1H, dt, J=8.8, 6.4 Hz, H-11a), 3.48 (1H, dt, J=8.8, 6.4 Hz, H-1′b), 1.48 (2H, m, H-2′), 1.34 (2H, m, H-3′), 0.91 (3H, t, J=7.2 Hz, CH₃-4′). ESI-MS m/z (rel. int.): 543[M+H]+. HR-ESI-MS: m/z 565.3137 [M+Na]⁺ (calculated for C₃₂H₄₆O₇Na, 565.3136).

The physical and chemical characters of compound Tubocapsanolide D (13): m.p.: 178˜180° C.; UV (MeOH): λₘₐₓ 220 nm; IR: νₘₐₓ 3491, 2937, 1687, 1382, 1132 cm⁻¹; ¹H NMR (400 MHz, CDCl₃): δ 6.03 (1H, dd, J=10.0, 2.4 Hz, H-2), 6.53 (1H, dd, J=10.0, 2.4 Hz, H-3), 5.02 (1H, s(br), H-4), 4.43 (1H, dd, J=12.8, 4.8 Hz, H-6), 1.64 (1H, m, H-7a), 2.01 (1H, ddd, J=9.6, 8.8, 4.8 Hz, H-7b), 1.63 (1H, d, J=8.8 Hz, H-8), 1.32 (1H, m, H-9), 1.01 (1H, m, H-11a), 1.56 (1H, m, H-11b), 1.37 (1H, m, H-12a), 1.56 (1H, m, H-12b), 1.26 (1H, m, H-14), 1.36 (1H, m, H-15a), 1.58 (1H, m, H-15b), 1.43 (1H, m, H-16a), 2.27 (1H, m, H-16b), 0.85 (3H, s, CH₃-18), 1.26 (3H, s, CH₃-19), 2.21 (1H, qd, J=7.2, 1.6 Hz, H-20), 1.08 (3H, d, J=7.2 Hz, CH₃-21), 4.61 (1H, ddd, J=12.8, 3.2, 1.6 Hz, H-22), 2.50 (1H, dd, J=18.0, 12.8 Hz, H-23a), 2.36 (1H, dd, J=18.0, 3.2 Hz, H-23b), 1.85 (3H, s, CH₃-27), 1.91 (3H, s, CH₃-28). FAB-MS m/z (rel. int.): 489[M+H]⁺. HR-ESI-MS: m/z 511.2668 [M+Na]⁺ (calculated for C₂₈H₄₀O₇Na, 511.2666).

The physical and chemical characters of compound Tubocapsanolide F (14): m.p.: 200˜202° C.; UV (MeOH): Sma 214 nm; IR: νₘₐₓ 3453, 2922, 1682, 1376, 1129, 750 cm⁻¹; ¹H NMR (400 MHz, C₅D₅N): δ 6.36 (1H, d, J=10.0 Hz, H-2), 7.17 (1H, dd, J=9.6, 6.4 Hz, H-3), 3.99 (1H, d, J=6.0, 3.2 Hz, H-4), 3.20 (1H, br s, H-6), 1.23 (1H, m, H-7a), 2.11 (1H, m, H-7b), 1.60 (1H, m, H-8), 1.01 (1H, m, H-9), 1.71 (1H, m, H-11a), 2.10 (1H, m, H-11b), 1.08 (1H, m, H-12a), 1.66 (1H, m, H-12b), 1.86 (1H, m, H-14), 1.75(1H, m, H-15a), 1.95(1H, m, H-15b), 1.89(1H, m, H-16a), 1.98(1H, m, H-16b), 0.73 (3H, s, H-18), 1.88 (3H, s, H-19), 2.31 (1H, qd, J=13.6, 6.8, 2.6 Hz, H-20), 1.19 (3H, d, J=6.8 Hz, H-21), 4.76 (1H, td, J=12.8, 9.6, 3.2 Hz, H-22), 2.42 (1H, t, J=18.0, 12.8 H-23a), 2.63 (1H, dd, J=18.0, 3.2, 2.8 Hz, H-23b), 1.93 (3H, s, H-27), 1.66 (3H, s, H-28). ESI-MS m/z (rel. int.): 493 [M+Na]⁺, 493(100), 453(12), 413(30), 381(65), 353(16). HR-ESI-MS: m/z 493.2564 [M+Na]⁺ (calculated for C₂₈H₃₈O₆Na, 493.2566).

The physical and chemical characters of compound Tubocapsanolide G (15): m.p.: 218˜220° C.; UV (MeOH): λₘₐₓ 206, 230 nm; IR: νₘₐₓ 3439, 2922, 1690, 1096, 750 cm⁻¹; ¹H NMR (400 MHz, C₅D₅N): δ 2.87 (1H, dd, J=15.5, 3.5 Hz, H-2a), 3.08 (1H, dd, J=15.5, 7.5 Hz, H-2b), 3.92 (1H, m, H-3), 3.90 (1H, br s, H-4), 3.37 (1H, br s, H-6), 1.37 (1H, m, H-7a), 2.21 (1H, dd, J=4.5, 2.5 Hz, H-7b), 1.61 (1H, m, H-8), 1.34 (1H, m, H-9), 1.08 (1H, m, H-11a), 1.72 (1H, m, H-11b), 1.73 (1H, m, H-12a), 1.97 (1H, m, H-12b), 1.92 (1H, m, H-14), 1.57 (1H, m, H-15a), 1.61 (1H, m, H-15b), 1.89 (1H, m, H-16a), 1.98 (1H, m, H-16b), 0.73 (3H, s, CH₃-18), 1.76 (3H, s, CH₃-19), 2.34 (1H, dq, J=7.0, 3.0 Hz, H-20), 1.20 (3H, d, J=7.0 Hz, CH₃-21), 4.77 (1H, td, J=13.0, 3.5, 3.0 Hz , H-22), 2.44 (1H, t, J=18.0, 13.0 Hz, H-23a), 2.65 (1H, dd, J=18.0, 3.5 Hz, H-23b), 1.94 (3H, s, CH₃-27), 1.65 (3H, s, CH₃-28), 3.29 (3H, s, OCH₃-1′). ESI-MS m/z (rel. int.): 525 [M+Na]⁺, 525(100), 413(29). HR-ESI-MS: m/z 525.2830 [M+Na]⁺ (calculated for C₂₉H₄₂O₇Na, 525.2828).

The physical and chemical characters of compound iso-Tubocapsanolide G (16): m.p.: 233˜235° C.; UV (MeOH): λₘₐₓ 207, 223 nm; IR: νₘₐₓ 3424, 2929, 1705, 1384, 1096, 753 cm⁻¹; ¹H NMR (400 MHz, C₅D₅N): δ 2.97 (1H, dd, J=15.2, 3.2 Hz, H-2a), 3.15 (1H, dd, J=15.2, 8.0 Hz, H-2b), 3.95 (1H, m, H-3), 3.92 (1H, d, J=2.4 Hz, H-4), 3.41 (1H, s(br), H-6), 1.40 (1H, m, H-7a), 2.19 (1H, m, H-7b), 1.56 (1H, m, H-8), 1.28 (1H, m, H-9), 1.50 (1H, m, H-11a), 1.54 (1H, m, H-11b), 1.23 (1H, m, H-12a), 1.96 (1H, m, H-12b), 1.85 (1H, m, H-14), 1.11 (1H, m, H-15a), 1.61 (1H, m, H-15b), 1.75 (1H, m, H-16a), 2.18 (1H, m, H-16b), 0.82 (1H, m, H-17), 0.97 (3H, s, CH₃-18), 1.71 (3H, s, CH₃-19), 1.39 (3H, s, CH₃-21), 4.38 (1H, dd, J=12.8, 3.6 Hz, H-22), 2.27 (1H, m, H-23a), 2.54 (1H, m, H-23b), 1.93 (3H, s, CH₃-27), 1.81 (3H, s, CH₃-28), 3.37 (3H, s, OCH₃-1′). ESI-MS m/z (rel. int.): 525 [M+Na]⁺, 525(100), 413(12). HR-ESI-MS: m/z 525.2830 [M+Na]⁺ (calculated for C₂₉H₄₂O₇Na, 525.2828).

The physical and chemical characters of compound 20-Hydroxytubocapsanolide G (17): m.p.: 20818 210° C.; UV (MeOH): λₘₐₓ 206, 228 nm; IR; νₘₐₓ 3461, 2922, 1705, 1384, 1089, 753 cm⁻¹; ¹H NMR (400 MHz, C₅D₅N): δ 2.91 (1H, dd, J=15.2, 3.2 Hz, H-2a), 3.09 (1H, dd, J=15.2, 8.0 Hz, H-2b), 3.91 (1H, m, H-3), 3.90 (1H, d, J=2.4 Hz, H-4), 3.37 (1H, br s, H-6), 1.39 (1H, m, H-7a), 2.19 (1H, m, H-7b), 1.65 (1H, m, H-8), 1.44 (1H, m, H-9), 1.64 (1H, m, H-11a), 1.69 (1H, m, H-11b), 1.90 (1H, m, H-12a), 2.16 (1H, m, H-12b), 2.03 (1H, dq, J=7.6, 3.6 Hz, H-14), 1.17 (1H, m, H-15a), 1.70 (1H, m, H-15b), 2.16 (1H, m, H-16a), 2.99 (1H, m, J=12.0, 3.2, 2.8 Hz, H-16b), 1.20 (3H, s, CH₃-18), 1.76 (3H, s, CH₃-19), 1.53 (3H, s, CH₃-21), 5.01(1H, m, H-22), 2.43(11H, dd, J=18.0, 3.2 Hz, H-23a), 2.73(1H, m, J=18.0, 14.8 Hz, H-23b), 1.85 (3H, s, CH₃-27), 1.67 (3H, s, CH₃-28), 3.30 (3H, s, OCH₃-1′). ESI-MS m/z (rel. int.): 541[M+Na]⁺, 541(100), 413(53), 381(55), 353(19). HR-ESI-MS: m/z 541.2780 [M+Na]⁺ (calculated for C₂₉H₄₂O₇Na, 541.2777).

The physical and chemical characters of compound Tubocapsanolide H (18): m.p.: 242˜244° C.; UV (MeOH): λₘₐₓ 205, 226 nm; IR: νₘₐₓ 3431, 2922, 2856, 1705, 1376, 1093, 750 cm⁻¹; ¹H NMR (400 MHz, C₅D₅N): δ 2.97 (1H, dd, J=15.6, 4.0 Hz, H-2a), 3.11 (1H, dd, J=15.6, 8.0 Hz, H-2b), 3.93 (1H, dd, J=8.0, 4.0, 3.2 Hz, H-3), 3.90 (1H, d, J=3.2 Hz,H-4), 3.40 (1H, br s, H-6), 1.39 (1H, m, H-7a), 2.18 (1H, m, H-7b), 1.42 (1H, m, H-9), 1.75 (1H, m, H-11a), 2.18 (1H, m, H-11b), 1.63 (1H, m, H-12a), 2.20 (1H, m, H-12b), 1.50 (1H, dq, H-14), 1.85 (1H, m, H-15a), 2.05 (1H, m, H-15b), 6.04 (1H, d, J=2.0 Hz, H-16), 1.03 (3H, s, CH₃-18), 1.73 (3H, s, CH₃-19), 1.55 (3H, s, CH₃-21), 4.55 (1H, dd, J=12.8, 3.6 Hz, H-22), 2.34 (1H, dd, J=18.0, 3.6 Hz, H-23a), 2.82 (1H, t, J=18.0 Hz, H-23b), 1.91 (3H, s, CH₃-27), 1.74 (3H, s, CH₃-28), 3.35 (3H, s, OCH₃-1′). ESI-MS m/z (rel. int.): 523 [M+Na]⁺, 523(100), 425(11), 413(16). HR-ESI-MS: m/z 523.2671 [M+Na]⁺ (calculated for C₂₉H₄₀O₇Na, 523.2672).

The physical and chemical characters of compound Anomanolide A (19): m.p.: 170˜172° C.; UV (MeOH): λₘₐₓ 214 nm; IR: νₘₐₓ 3432, 2952, 1720, 1675, 1380, 1128, 754 cm⁻¹; ¹H NMR (400 MHz, CDCl₃): δ 6.19 (1H, d, J=10.0 Hz, H-2), 6.93 (1H, dd, J=10.0, 6.0 Hz, H-3), 3.76 (1H, d, J=6.0 Hz, H-4), 3.24 (1H, s(br), H-6), 1.35 (1H, dd, J=15.2, 3.2 Hz, H-7a), 2.16 (1H, ddd, J=15.2, 4.0, 3.2 Hz, H-7b), 1.55 (1H, td, J=10.8, 4.0 Hz, H-8), 1.01 (1H, td, J=10.8, 3.2 Hz, H-9), 1.37 (1H, m, H-11a), 1.92 (1H, m, H-11b), 1.42 (2H, m, H-12a,b), 1.43 (1H, m, H-14), 1.23 (1H, m, H-15a), 1.73 (1H, m, H-15b), 1.70 (1H, m, H-16a), 2.04 (1H, d, J=8.4 Hz, H-16b), 0.75 (3H, s, CH₃-18), 1.40 (3H, s, CH₃-19), 2.43 (1H, ddd, J=9.2, 7.6, 1.0 Hz, H-20), 1.31 (1H, dd, J=12.8, 7.6 Hz, H-21a), 2.51 (1H, ddd, J=12.8, 9.2, 2.0 Hz, H-21b), 4.66 (1H, d, J=2.4 Hz, H-22), 2.07 (1H, d, J=13.2 Hz, H-23a), 1.78 (1H, dd, J=13.2, 2.4 Hz, H-23b), 1.45 (3H, s, CH₃-27), 1.18 (3H, s, CH₃-28). FAB-MS m/z (rel. int.): 487 [M+H]⁺. HR-ESI-MS: m/z 509.2517 [M+Na]⁺ (calculated for C₂₈H₃₈O₇Na, 509.2510).

The physical and chemical characters of compound Anomanolide B (20): m.p.: 168˜170° C.; UV (MeOH): λₘₐₓ 214, 230 nm; IR: νₘₐₓ 3448, 2960, 1726, 1675, 1369, 1130 cm⁻¹; ¹H NMR (400 MHz, CDCl₃): δ 5.98 (1H, dd, J=10.0, 2.0 Hz, H-2), 6.45 (1H, dd, J=10.0, 2.0 Hz, H-3), 5.03 (1H, s(br), H-4), 4.42 (1H, dd, J=12.8, 4.4 Hz, H-6), 1.72 (1H, m, H-7a), 2.32 (1H, 2.32 dt, J=9.6, 4.4 Hz, H-7b), 1.65 (1H, m, H-8), 1.28 (1H, dd, J=9.6, 3.2 Hz, H-9), 0.96 (1H, d, J=8.0 Hz, H-11a), 1.32 (1H, m, H-11b), 1.35 (1H, m, H-12a), 1.39 (1H, m, H-12b), 1.59 (1H, m, H-14), 1.27 (1H, m, H-15a), 1.73 (1H, m, H-15b), 1.66 (1H, m, H-16a), 2.08 (1H, m, H-16b), 0.72 (3H, s, CH₃-18), 1.24 (3H, s, CH₃-19), 2.40 (1H, q, J=7.6 Hz, H-20), 1.25 (1H, m, H-21a), 2.44 (1H, dd, J=12.8, 2.4 Hz, H-21b), 4.64 (1H, d, J=2.4 Hz, H-22), 2.05 (1H, d, J=14.8 Hz, H-23a), 1.74 (1H, dd, J=14.8, 2.4 Hz, H-23b), 1.43 (3H, s, CH₃-27), 1.15 (3H, s, CH₃-28). ESI-MS m/z (rel. int.): 505 [M+H]⁺.

The physical and chemical characters of compound Anomanolide C (21): m.p.: 280˜282° C.; UV (MeOH): λₘₐₓ 214 nm; IR: νₘₐₓ 3430, 2927, 1702, 1677, 1369, 1130 cm⁻¹; ¹H NMR (400 MHz, C₅D₅N): δ 6.38 (1H, d, J=10.0 Hz, H-2), 7.18 (1H, dd, J=10.0, 6.4 Hz, H-3), 3.98 (1H, dd, J=6.4, 2.4 Hz, H-4), 3.16 (1H, s(br), H-6), 1.20 (1H, dd, J=14.0, 11.2 Hz, H-7a), 2.04 (1H, m, H-7b), 1.51 (1H, m, H-8), 1.02 (1H, ddd, J=11.2, 4.0, 2.8 Hz, H-9), 1.51 (1H, m, H-11a), 2.04 (1H, m, H-11b), 1.58 (1H, m, H-12a), 2.01 (1H, m, H-12b), 1.98 (1H, m, H-14), 1.67 (1H, m, H-15a), 1.75 (1H, dd, J=13.6, 7.2 Hz, H-15b), 4.51 (1H, dd, J=7.2, 3.2 Hz, H-16), 0.63 (3H, s, CH₃-18), 1.80 (3H, s, CH₃-19), 2.75 (1H, dd, J-=8.4, 8.0 Hz, H-20), 1.98 (1H, m, H-21a), 2.99 (1H, dd, J=9.6, 8.0 Hz, H-21b), 5.47 (1H, d(br), J=2.0 Hz, H-22), 2.15 (1H, d, J=12.8 Hz, H-23a), 2.04 (1H, dd, J=12.8, 2.8 Hz, H-23b), 1.67 (3H, s, CH₃-27), 1.34 (3H, s, CH₃-28). EI-MS m/z (rel. int.): 502 [M]⁺. HR-ESI-MS: m/z 525.2466 [M+Na]⁺ (calculated for C₂₈H₃₈O₈Na, 525.2459).

The physical and chemical characters of compound Anomanolide D (22): m.p.: 196˜198° C.; UV (MeOH): λₘₐₓ 214 nm; IR:. νₘₐₓ 3448, 2948, 1718, 1675, 1378, 1126, 1049 cm⁻¹; ¹H NMR (400 MHz, C₅D₅N): δ 6.16 (1H, dd, J=10.0, 2.0 Hz, H-2), 6.77 (1H, dd, J=10.0, 2.0 Hz, H-3), 5.19 (1H, s(br), H-4), 4.64 (1H, dd, J=12.8, 4.4 Hz, H-6), 1.82 (1H, t, J=12.0 Hz, H-7a), 2.21 (1H, dt, J=12.8, 4.0 Hz, H-7b), 1.59 (1H, m, H-8), 1.59 (1H, m, H-9), 1.10 (1H, m, H-11a), 1.37 (1H, m, H-11b), 1.46 (1H, dt, J=9.6, 2.8 Hz, H-12a), 1.94 (1H, m, H-12b), 2.12 (1H, m, H-14), 1.72 (2H, d, J=11.2 Hz, H-15a,b), 4.50(11H, d(br), J=3.2 Hz, H-16), 0.61(3H, s, CH₃-18), 1.53(3H, s, CH₃-19), 2.71 (1H, t, J=8.4 Hz, H-20), 1.93 (1H, m, H-21a), 2.95 (1H, dd, J=11.2, 9.6 Hz, H-21b), 5.45 (1H, d, J=2.0 Hz, H-22), 2.14 (1H, d, J=12.8 Hz, H-23a), 2.02 (1H, dd, J=12.8, 3.2 Hz, H-23b), 1.66 (3H, s, CH₃-27), 1.34 (3H, s, CH₃-28). FAB-MS m/z (rel. int.): 539[M+H]⁺. HR-ESI-MS: m/z 561.2229 [M+Na]⁺ (calculated for C₂₉H₃₉ClO₈Na, 561.2226).

The physical and chemical characters of compound Anomanolide E (23): m.p.: 182˜184° C.; UV (MeOH): λₘₐₓ 214 nm; IR: νₘₐₓ 3455, 2933, 1720, 1675, 1400, 1132 cm⁻; ¹H NMR (400 MHz, C₅D₅N): δ 6.12 (1H, dd, J=10.0, 2.4 Hz, H-2), 6.64 (1H, ddd, J=10.0, 3.6, 2.4 Hz, H-3), 2.39 (1H, d, J=19.2 Hz, H-4eq), 3.73 (1H, ddd, J=19.6, 2.4, 2.4 Hz, H-4ax), 4.10 (1H, s(br), H-6), 1.85 (1H, dt, J=12.0, 2.8 Hz, H-7a), 2.23 (1H, td, J=12.0, 2.4 Hz, H-7b), 2.14 (1H, m, H-8), 2.56 (1H, td, J=11.2, 3.2 Hz, H-9), 1.58 (1H, qd, J=12.0, 3.2 Hz, H-11a), 2.83 (1H, dt, J=12.0, 3.2 Hz, H-11b), 1.71 (1H, m, H-12a), 2.41 (1H, m, H-12b), 2.41 (1H, m, H-14), 1.79 (2H, m, H-15a,b), 4.49 (1H, d, J=6.4 Hz, H-16), 0.76 (3H, s, CH₃-18), 1.67 (3H, s, CH₃-19), 2.79 (1H, dd, J=8.4, 8.0 Hz, H-20), 2.04 (1H, dd, J=12.8, 7.6 Hz, H-21a), 3.02 (1H, ddd, J=12.8, 9.6, 1.2 Hz, H-21b), 5.47 (1H, d, J=2.0 Hz, H-22), 2.15 (1H, d, J=12.0 Hz, H-23a), 2.07 (1H, dd, J=12.0, 2.8 Hz, H-23b), 1.67 (3H, s, CH₃-27), 1.36 (3H, s, CH₃-28). FAB-MS m/z (rel. int.): 505 [M+H]⁺. HR-ESI-MS: m/z 527.2618 [M+Na]⁺ (calculated for C₂₈H₄₀O₈Na, 527.2615).

The physical and chemical characters of compound Anomanolide F (24): m.p.: 190˜192° C.; UV (MeOH): λₘₐₓ 215 nm; IR: νₘₐₓ 3416, 2929, 2863, 1712, 1668, 1376, 1082, 753 cm⁻¹; ¹H NMR (400 MHz, C₅D₅N): δ 6.12 (1H, dd, J=10.4, 2.4 Hz, H-2), 6.73 (1H, dd, J=10.4, 2.4 Hz, H-3), 5.35 (1H, br s, H-4), 1.22 (1H, m, H-6a), 2.22 (1H, m, H-6b), 1.24 (1H, m, H-7a), 1.78 (1H, m, H-7b), 1.65 (1H, m, H-8), 1.56 (1H, m, H-9), 1.38 (2H, m, H-11ab), 1.54 (1H, m, H-12a), 1.99 (1H, m, H-12b), 2.16 (1H, m, H-14), 2.09 (2H, m, H-15ab), 4.42 (1H, s(br), H-16), 0.71 (3H, s, CH₃-18), 1.61 (3H, s, CH₃-19), 2.55 (1H, t, J=8.8, 8.4 Hz, H-20), 1.96 (1H, m, H-21a), 2.71 (1H, d, J=12.0 Hz, H-21b), 5.00 (1H, m, H-22), 2.02 (1H, m, H-23a), 2.05 (1H, m, H-23a), 1.81 (3H, S, CH₃-27), 1.40 (3H, s, CH₃-28). ESI-MS m/z (rel. int.): 527 [M+Na]⁺, 527(100), 413(61). HR-ESI-MS: m/z 527.2620 [M+Na]⁺ (calculated for C₂₈H₄₀O₈Na, 527.2621).

The physical and chemical characters of compound Tubonolide A (25): m.p.: 200˜202° C.; UV (MeOH): λₘₐₓ 214 nm; IR: νₘₐₓ 3427, 2938, 1730, 1676, 1371, 985, 752 cm⁻¹; ¹H NMR (400 MHz, C₅D₅N): δ 6.25 (1H, dd, J=8.0, 1.2 Hz, H-2), 6.83 (1H, dd, J=8.0, 2.0 Hz, H-3), 5.23 (1H, m(br), H-4), 4.69 (1H, dd, J=10.0, 4.0 Hz, H-6), 1.88 (1H, dd, J=10.0, 10.0 Hz, H-7a), 2.26 (1H, ddd, J=10.0, 4.0, 3.2 Hz, H-7b), 1.59 (1H, m, H-8), 1.56 (1H, td, J=8.8, 3.2 Hz, H-9), 1.09 (1H, m, H-11a), 1.43 (1H, m, H-11b), 1.41 (1H, m, H-12a), 1.83 (1H, m, H-12b), 2.13 (1H, m, H-14), 1.72 (1H, m, H-15a), 1.79 (1H, m, H-15b), 4.49 (1H, t, J=4.4 Hz, H-16), 0.75 (3H, s, CH₃-18), 1.62 (3H, s, CH₃-19), 2.62 (1H, m, H-20), 2.67 (1H, dd, J=10.4, 5.6 Hz, H-21ax), 1.64 (1H, d, J=10.4 Hz, H-21eq), 5.09 (1H, s(br), H-22), 2.91 (1H, d, J=12.4 Hz, H-23ax), 2.15 (1H, m, H-23b), 1.50 (3H, s, CH₃-27), 1.36 (3H, s, CH₃-28), 7.43 (1H, d, J=5.6 Hz, 4-OH), 5.99 (1H, s, 5-OH), 7.28 (1H, d, J=3.6 Hz, 16-OH), 5.41 (1H, s, 17-OH), 6.11 (1H, s, 24-OH). ESI-MS m/z (rel. int.): 539[M+H]⁺. HR-ESI-MS: m/z 561.2232 [M+Na]⁺ (calculated for C₂₈H₃₉ClO₈Na, 561.2226).

Bioactivity Tests

The bioactivities of the 25 new withanolide compounds of the present invention are further studied. The bioactivities are measured by examining cytotoxicity tests with 5 human cancer cell lines, including MCF-7 and MDA-MB-231 (which are the breast cancer cell lines), Hep G2 and Hep 3B (which are the liver cancer cell line), and A549 (which is lung cancer cell line). In addition, a normal human lung cell line (MRC-5) is served as the control group. All human cancer cell lines come from the American Type Culture Collection. The respective cell lines are cultured in RPMI-1640 medium with 10% (v/v) fetal calf serum, 100 U/ml penicillin and 100 g/ml streptomycin, and incubated at 37° C. in 5% CO₂ atmosphere.

In the present invention, the cytotoxicity tests are performed with MTT (3-(4,5-dimethyl-thiazol-2-yl)-2,5-diphenyl tetrazolium bromide) method. The five cancer cell lines and the normal cell line (MRC-5) are firstly incubated in a 96-well culture dish with concentrations of 5,000˜10,000 cells/well. In Day 2, the respective incubated cells are added with the withanolide compounds of the present invention and Doxorubicin and are incubated for additional 72 hours. Afterwards, the incubated cells are examined with the MTT method including steps of dissolving the incubated cells in DMSO, and determining the absorbance of each cell line to each compound by a microplate reader in 550 nm to evaluate the cytotoxicity of each compound to each cell line. The examining results are showed in table 1, which shows the cytotoxicities of each withanolide compound in the present invention. The IC₅₀ represents the concentration of the compound that is required for 50% inhibition of the cell growth, and Doxorubicin serves as the positive control group. As the table 1 shows, the withanolide compounds 1, 8, 9, 10 and 14 of the present invention have great cytotoxicities in 5 target cancer cell lines, where the IC₅₀ are small than 2 μg/ml.

Based on the description above, the composition for treating cancer cells and the preparation method therefore in the present invention not only provides the new withanolide compounds that are extracted from Tubocapsicum anomalum, and more particularly the new withanolide compounds have cytotoxicity to the cancer cells.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

TABLE 1
Cytotoxicities of the withanolide compounds 1-25

| | Cell lines (IC₅₀, μg/ml) | | | | | |
|---|---|---|---|---|---|---|
| | | | | | MDA- | |
| | | | | | MB- | |
| compounds | Hep G2 | Hep 3B | MCF-7 | A-549 | 231 | MRC-5 |
| 1 | 0.44 | 0.26 | 0.97 | 0.15 | 0.13 | 0.20 |
| 3 | 15.93 | >20 | >20 | 7.06 | >20 | |
| 4 | 4.57 | 2.30 | 6.77 | 4.24 | 6.55 | 4.24 |
| 5 | 12.31 | 13.20 | >20 | 15.94 | 17.50 | |
| 6 | >20 | 15.07 | 14.71 | 6.04 | 8.41 | |
| 7 | 19.12 | >20 | >20 | 19.94 | >20 | |
| 8 | 0.86 | 0.42 | 1.47 | 0.47 | 0.22 | 0.73 |
| 9 | 0.73 | 0.99 | 1.77 | 1.42 | 0.99 | 1.36 |
| 10 | 0.44 | 0.49 | 2.05 | 0.79 | 1.19 | 0.90 |
| 13 | >20 | 7.19 | >20 | 8.01 | 13.49 | |
| 14 | 0.64 | 0.80 | 1.98 | 0.88 | 0.99 | 0.81 |
| 15 | 17.55 | >20 | >20 | >20 | >20 | |
| 16 | 5.99 | 7.63 | >20 | 4.73 | 8.18 | |
| 17 | 2.51 | 12.30 | 17.25 | 2.67 | 4.44 | 7.20 |
| 18 | 3.67 | 2.65 | 9.27 | 7.50 | 4.82 | 15.23 |
| 19 | 3.11 | 3.63 | 2.31 | 1.01 | 1.37 | 1.91 |
| 20 | 0.97 | 3.17 | 4.84 | 1.49 | 0.70 | 0.20 |
| 21 | 4.41 | 1.85 | 8.01 | 2.80 | 1.58 | |
| 22 | >20 | 19.03 | >20 | >20 | >20 | |
| 23 | >20 | >20 | >20 | >20 | >20 | |
| 24 | >20 | >20 | >20 | >20 | >20 | |
| 25 | 5.09 | 6.54 | 12.03 | 5.91 | >20 | |
| withaferin A | 0.06 | 0.06 | 0.05 | 0.02 | 0.02 | 0.07 |
| doxorubicin | 0.46 | 0.45 | 0.34 | 0.42 | 0.19 | 0.77 |

What is claimed is:

1. A cytotoxic composition, comprising a withanolide compound having a structure selected from a group consisting of the following formulas: 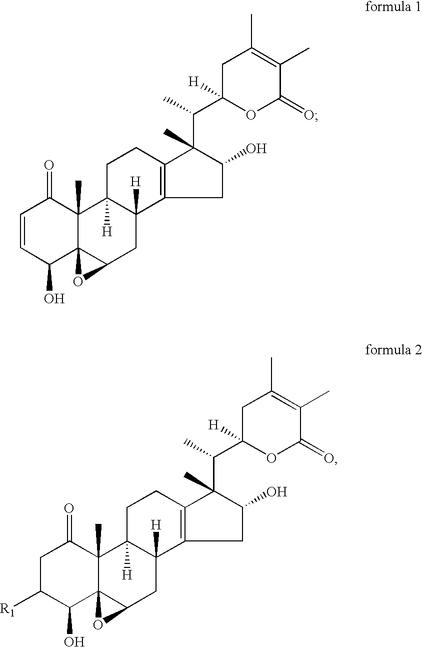
wherein R₁ is one selected from a group consisting of OH and C1-C4 alkoxy groups; 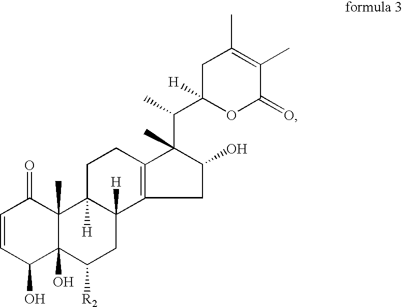
wherein R₂ is one selected from a group consisting of H, OH, halogen and C1-C4 alkoxy groups; 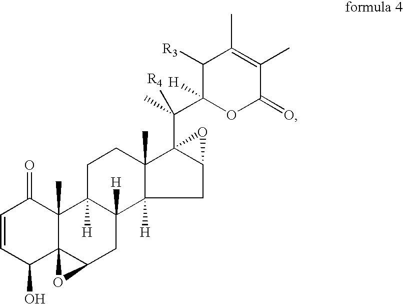
wherein each of R₃ and R₄ is selected from one of H and OH; 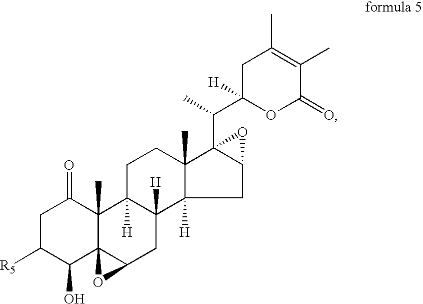
wherein R₅ is one selected from a group consisting of OH and C1-C4 alkoxy groups; and

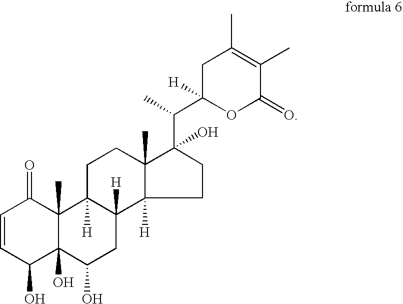

2. A cytotoxic composition as claimed in claim 1, wherein the withanolide compound is extracted from a Solanaceae plant.

3. A cytotoxic composition as claimed in claim 2, wherein the solanaceae plant is a Tubocapsicum anomalum Makino.

4. A cytotoxic composition as claimed in claim 1 further comprising one of a pharmaceutically acceptable carrier and an excipient.

5. A cytotoxic composition as claimed in claim 1, wherein the cytotoxic composition is used for treating a cancer.

6. A cytotoxic composition as claimed in claim 5, wherein the cancer is one selected from a group consisting of a lung cancer, a liver cancer, and a breast cancer.

7. A cytotoxic composition, comprising a withanolide compound having a structure selected from a group consisting of the following formulas: 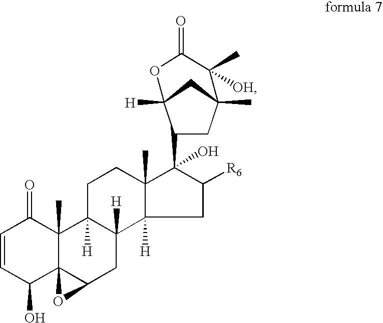
wherein R₆ is one of H and OH; 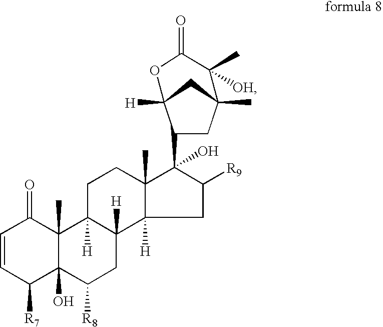
wherein each of R₇ and R₉ is one of H and OH, and R8 is one selected from a group consisting of H, OH and halogen; 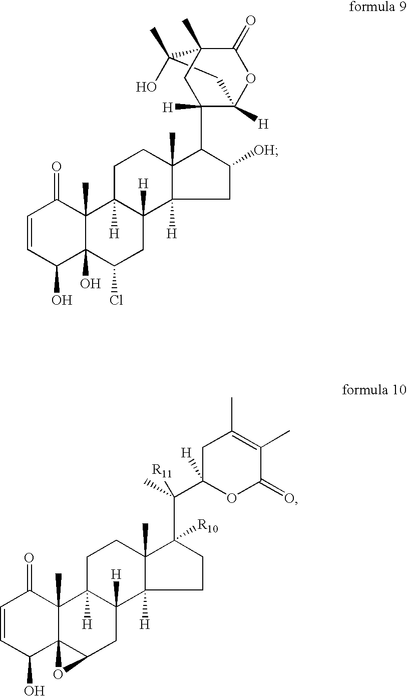
wherein each of R₁₀ and R₁₁ is one of H and OH; 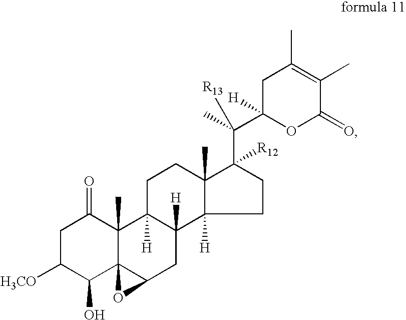
wherein each of R₁₂ and R₁₃ is one of H and OH; and

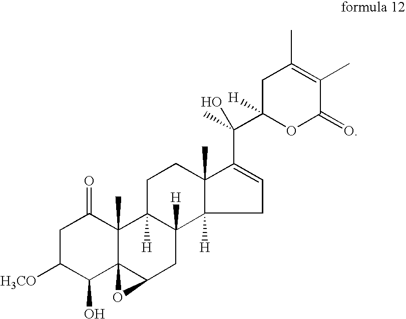

8. A cytotoxic composition as claimed in claim 7, wherein the withanolide compound is extracted from a Solanaceae plant.

9. A cytotoxic composition as claimed in claim 8, wherein the Solanaceae plant is a Tubocapsicum anomalum.

10. A cytotoxic composition as claimed in claim 7 further comprising one of a pharmaceutically acceptable carrier and an excipient.

11. A cytotoxic composition as claimed in claim 7, wherein the cytotoxic composition is used for treating a cancer.

12. A cytotoxic composition as claimed in claim 11, wherein the cancer is one selected from a group consisting of a lung cancer, a liver cancer, and a breast cancer.

13. A method for preparation of a withanolide compound, comprising steps of:
providing a Tubocapsicum anomalum;
extracting the Tubocapsicum anomalum with a first organic solvent to obtain a first extract;
extracting the first extract with a second organic solvent to obtain a second extract; and
isolating the second extract to obtain the withanolide compound.

14. A method as claimed in claim 13 further comprising a step of extracting the second extract with a third organic solvent to obtain a third extract.

15. A method as claimed in claim 14, wherein the third organic solvent is an n-butanol.

16. A method as claimed in claim 14 further comprising a step of isolating the third extract to obtain the withanolide compound.

17. A method as claimed in claim 16, wherein the withanolide compound is isolated from the third extract by a chromatography method.

18. A method as claimed in claim 13, wherein the first organic solvent is a methanol.

19. A method as claimed in claim 13, wherein the second organic solvent is an ethyl acetate.

20. A method as claimed in claim 13, wherein the withanolide compound is isolated from the second extract by a chromatography method.
